(19) United States
(12) Patent Application Publication
(10) Pub. No.: US 2004/0142384 A1
(43) Pub. Date: Jul. 22, 2004

(54) Magnetic separator

(76) Inventors: Barb Ariel Cohen, Watertown, MA (US); Barbara J. Hughey, Lexington, MA (US); Michael F. Morris, Ashland, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/346,576

(22) Filed: Jan. 16, 2003

(51) Int. Cl. G01N 33/53

(52) U.S. Cl. 435/7.2

(57) ABSTRACT

This invention relates to magnetic separators for magnetically separating different components of a test sample. The magnetic separators can be used in methods of separating cells.

### FIELD OF THE INVENTION

This invention relates to magnetic separators and methods of separating cells using magnetic separation. More particularly, this invention relates to magnetic separators and methods of separation of spermatozoa determinative of one sex from spermatozoa of the other sex.

### BACKGROUND OF THE INVENTION

Farmers and other animal husbandry persons have long recognized the desirability of enhancing the probability of obtaining offspring of a selected sex. Methods have been proposed in the past for increasing the percentage of X-chromosome bearing sperm cells or Y-chromosome bearing sperm cells to thereby achieve a greater chance of achieving female or male offspring, respectively.

Previous methods have included, for example, methods based upon density sedimentation (see, for example, Brandriff, B. F. et al. “Sex Chromosome Patios Determined by Karyotypic Analysis in Albumin-Isolated Human Sperm,” Fertil. Steril., 46, pp. 678-685 (1986)).

U.S. Pat. No. 3,687,806 to Van Den Bovenkamp discloses an immunological method for controlling the sex of mammalian offspring by use of antibodies which react with either X-bearing sperm or Y-bearing sperm and utilizing an agglutination step to separate bound antibodies from unaffected antibodies.

U.S. Pat. No. 4,191,749 to Bryant discloses a method for increasing the percentage of mammalian offspring of either sex by use of a male-specific antibody coupled to a solid-phase immunoabsorbant material to selectively bind male-determining spermatozoa, while the female-determining spermatozoa remain unbound in a supernatant.

U.S. Pat. No. 5,021,244 to Spaulding discloses a method for sorting living cells based upon DNA content, particularly sperm populations to produce subpopulations enriched in X- or Y-sperm by means of sex-associated membrane proteins and antibodies specific for such proteins.

However, these methods often result in insufficient separation of X- and Y-sperm and often damage the sperm, thereby reducing its motility and fertility success rate.

In commonly assigned U.S. Pat. Nos. 6,153,373 and 6,489,092, improved methods using antibodies coupled to magnetic particles for separation of spermatozoa are provided. These methods, while providing gentle separation of populations of spermatozoa, use magnetic separation in a device that requires aspiration or decantation of supernatant, i.e., the materials not bound to magnetic particles and thus held by the magnetic field.

Other magnetic separators also require that the user aspirate or decant sample from the separator, which tends to mix the sample with the cells that are separated via binding to magnetic beads. Certain magnetic separator devices have attempted to overcome the problem of mixing by applying a magnetic field of high field strength to hold the cells bound by magnetic particles tightly to the walls of the separator such that no mixing occurs when the non-bound sample is removed from the separator. This approach has drawbacks, including the difficulty of applying high field strength external magnetic fields and the deleterious effects of high field strength magnetic fields on cells, particularly the effects of devices (e.g., steel wool) used to create high field strength internal magnetic fields.

Therefore, there is a need for a magnetic separation device that can efficiently separate cells without damaging the cells.

### SUMMARY OF THE INVENTION

The invention provides magnetic separators that overcome the difficulties of inefficient separation and damage to separated cells that existed with previous magnetic separation devices. The invention also provides methods of separating cells using the magnetic separator, populations of separated cells and methods for insemination using the populations of separated cells. The invention also provides methods for fractionating ejaculates based on an unexpected criticality of time and temperature in certain aspects of the separation process.

According to one aspect of the invention, a magnetic separator for separating magnetic components from a test sample that includes the magnetic components and non-magnetic components is provided. The magnetic separator includes a container constructed and arranged to receive the test sample, the container including an inlet and an outlet, the test sample to be received through the inlet; at least one magnet adapted to generate a magnetic field within the container, the magnetic field to be operative upon the magnetic components within the test sample to substantially separate the magnetic and non-magnetic components from one another; and a regulator coupled to the outlet of the container to regulate flow of the non-magnetic components from the outlet of the container.

In some embodiments, the regulator is actuatable between a closed position and an open position to control the flow of the non-magnetic components from the outlet. Preferably the regulator is actuatable to vary the rate of flow of the non-magnetic components from the outlet. In other embodiments, the regulator includes a valve; preferably the valve includes a stopcock.

In further embodiments, the outlet is located below the inlet. Preferably the outlet of the container is provided at a bottom of the container. More preferably, the bottom of the container has a substantially conical shape.

In still other embodiments, at least a portion of the container is substantially transparent such that the test sample within the container is visible from outside the container.

In certain embodiments of the invention, the at least one magnet includes a bar magnet and/or includes a pair of magnets that are spaced apart about the container. Preferably the magnets are substantially equally spaced about the container; more preferably, the magnets are spaced approximately 180° apart about the container. The magnet is formed of a material selected from the group consisting of neodymium iron boron, samarium cobalt, alnico and ferrite in some embodiments. In other embodiments, the magnet includes at least one electromagnet.

The invention in still other embodiments also includes at least one retainer constructed and arranged to hold the container adjacent the magnet. Preferably, the at least one retainer slidably receives the container. In another preferred embodiment, the at least one retainer includes a channel constructed and arranged to receive a portion of an outer surface of the container.

According to another aspect of the invention, a magnetic separator for separating magnetic components from a test sample that includes the magnetic components and non-magnetic components is provided. The magnetic separator includes a container-receiving region that is constructed and arranged to receive a container that is adapted to receive the test sample; at least two magnets spaced about the container-receiving region, the magnets adapted to generate a magnetic field within the container-receiving region; and a guide constructed and arranged to position the container within the container-receiving region at a substantially equal distance from each magnet.

In some embodiments, the guide includes at least one retainer constructed and arranged to hold the container, and/or the guide includes at least one channel constructed and arranged to receive at least a portion of an outer surface of the container, and/or the guide is constructed and arranged to slidably receive the container or is constructed and arranged to receive the container by a snap-fit configuration.

In other embodiments, the magnets are substantially equally spaced about the container-receiving region. The magnets are formed of a material selected from the group consisting of neodymium iron boron, samarium cobalt, alnico and ferrite in certain embodiments. In still other embodiments, the magnetic separator is provided in combination with the container, the container being positioned in the container-receiving region by the guide at a substantially equal distance from each magnet.

According to a further aspect of the invention, a magnetic separator for separating magnetic components from a test sample that includes the magnetic components and non-magnetic components is provided. In this aspect of the invention, the separator includes a container-receiving region that is constructed and arranged to receive a container that is adapted to receive the test sample; at least one magnet disposed adjacent the container-receiving region, the magnet adapted to generate a magnetic field within the container-receiving region, the magnetic field to be operative upon the magnetic components in the test sample; and a base supporting the container-receiving region above a vessel-receiving region that is constructed and arranged to receive a vessel below the container-receiving region, the vessel adapted to capture the non-magnetic components of the test sample from the container.

In certain embodiments, the base includes a plurality of legs adapted to elevate the container-receiving region, and/or the base is securable to a surface. The magnetic separator in other embodiments also includes at least one retainer constructed and arranged to hold the container in the container-receiving region. Preferably the retainer maintains the magnet spaced a distance from the container-receiving region.

In further embodiments, the at least one magnet includes a pair of magnets that are substantially equally spaced about the container-receiving region. In still other embodiments, the at least one magnet includes a bar magnet. The magnet is formed of a material selected from the group consisting of neodymium iron boron, samarium cobalt, alnico and ferrite in still other embodiments. The magnet can include at least one electromagnet.

In some embodiments, the magnetic separator is provided in combination with the container, the container being positioned in the container-receiving region and being adapted to receive the test sample. In some of these embodiments, the magnetic separator is provided in combination with the vessel, the vessel being positioned in the vessel-receiving region and adapted to capture the non-magnetic components from the container. Preferably the container includes an outlet constructed and arranged for the non-magnetic components to flow out of the container from the outlet. In certain of these preferred embodiments, the vessel is provided to receive the flow of the non-magnetic components from the outlet of the container and/or the outlet includes a regulator to regulate flow of the non-magnetic components from the outlet of the container.

According to yet another aspect of the invention, methods for magnetically separating a selected population of cells from a biological sample are provided. The methods include contacting the biological sample in a container with a plurality of binding agent molecules that selectively bind the selected population of cells, for a time sufficient for the binding agent molecules to bind the cells, wherein the binding agent molecules are attached to magnetic particles, to form a magnetic component of the biological sample. The methods further include applying an external magnetic field to the container to separate the magnetic component from the non-magnetic components of the biological sample; and draining the non-magnetic components of the biological sample from the container to separate the selected population of cells from the non-magnetic components of the biological fluid sample. In some of these methods, the biological sample comprises a second population of cells and the non-magnetic components of the biological fluid sample include the second population of cells.

In certain embodiments, the binding agent molecule is an antibody or antigen-binding fragment thereof. Preferably the antibody is specific for Y-bearing sperm or for X-bearing sperm. In other embodiments, the antibody is attached to the magnetic particles through an intermediate linking compound. Preferably the intermediate linking compound is Protein A.

In still other embodiments, the binding agent molecule is a phage display binding molecule, a lectin or a binding partner of a molecule on the cell.

In still further embodiments, the magnetic particle is a non-porous magnetic bead support, preferably one having a diameter of 0.1 to 2 microns, more preferably having a diameter of 0.1 to 0.5 microns. In certain of the foregoing embodiments, the magnetic particle is covalently attached to the binding agent molecule.

In the methods of the invention, the selected population of cells preferably is spermatozoa determinative of one sex.

In some of the foregoing methods, the magnetic field is insufficient to hold the magnetic particles to the surface of the container. In certain preferred embodiments, the selected population of cells bound to the magnetic particles form a phase separate from the remainder of the biological fluid sample, and/or the selected population of cells bound to the magnetic particles form a bolus upon draining, the bolus protruding from the interior surface of the container.

In other preferred embodiments, the magnetic particles are too numerous to form a monolayer of particles on the walls of the container under the influence of the magnetic field. In further embodiments, the number of cells in the selected population of cells is greater than about 1×10⁵ cells/ml.

The methods can also include removing the selected population of cells from the container in certain embodiments. In preferred embodiments, the step of draining the selected population of cells from the container comprises draining the container by gravity, preferably by regulating the opening and optional closing of a valve or stopcock, or by regulating the operation of a pump attached to a drain. In alternative embodiments, the step of draining the selected population of cells from the container comprises pumping a dense fluid into the container to displace the non-magnetic components of the biological sample from the container.

In a further aspect of the invention, methods of insemination are provided. The methods include obtaining a population of spermatozoa according to any of the methods described herein, and inseminating a mammal with the population of spermatozoa.

Methods for magnetically separating a selected population of cells from a biological sample are provided in another aspect of the invention. The methods include contacting the biological fluid sample with a binding agent that selectively binds the selected population of cells for a time sufficient for the binding agent to bind the selected population of cells to form a reaction mixture, wherein the binding agent is attached to a magnetic particle; transferring the reaction mixture to a separation container; applying an external magnetic field to the separation container to separate the magnetic particles from the biological fluid sample; and draining the non-magnetic components of the biological sample from the container to separate the selected population of cells from the non-magnetic components of the biological fluid sample.

In some embodiments, the biological sample comprises a second population of cells and the non-magnetic components of the biological fluid sample comprise the second population of cells.

In certain embodiments, the binding agent molecule is an antibody or antigen-binding fragment thereof. Preferably the antibody or antigen-binding fragment thereof is specific for Y-bearing sperm or for X-bearing sperm. In other preferred embodiments, the antibody is attached to the magnetic particles through an intermediate linking compound, which preferably is Protein A. In other embodiments, the binding agent molecule is a phage display binding molecule, a lectin, or a binding partner of a molecule on the cell.

The magnetic particle used in the methods preferably is a non-porous magnetic bead support, preferably having a diameter of 0.1 to 2 microns, more preferably having a diameter of 0.1 to 0.5 microns. In certain of the foregoing methods, the magnetic particle is covalently attached to the binding agent molecule.

In a particularly preferred embodiment, the selected population of cells is spermatozoa determinative of one sex.

The magnetic field in some embodiments is insufficient to hold the magnetic particles to the surface of the container. In certain of these embodiments, the selected population of cells bound to the magnetic particles form a phase separate from the remainder of the biological fluid sample and/or form a bolus upon draining that protrudes from the interior surface of the container. In other of these embodiments, the magnetic particles are too numerous to form a monolayer of particles on the walls of the container under the influence of the magnetic field.

The methods of the invention provide for efficient and gentle separation of populations of cells. In some of the methods, the number of cells in the selected population of cells is greater than about 1×10⁵ cells/ml.

In further embodiments, the methods also include removing the selected population of cells from the container. In some embodiments, removing the selected population of cells from the container includes a step of draining the selected population of cells from the container. Preferably, the step of draining includes draining the container by gravity. In certain preferred embodiments, the step of draining is regulated by opening and optionally closing a valve or stopcock, and/or regulating the operation of a pump attached to a drain. In alternative embodiments, the step of draining the selected population of cells from the container includes pumping a dense fluid into the container to displace the non-magnetic components of the biological sample from the container.

According to yet another aspect of the invention, methods of insemination are provided. The methods include obtaining a population of spermatozoa using the foregoing methods of separating populations of cells, and inseminating a mammal with the population of spermatozoa.

Methods of increasing the percentage of mammalian offspring of either sex are provided in another aspect of the invention. The methods include magnetically separating spermatozoa determinative of one sex from a biological sample containing spermatozoa of determinative of both sexes by contacting the biological fluid sample in a container with a plurality of binding agent molecules that selectively bind the spermatozoa determinative of one sex, for a time sufficient for the binding agent molecules to bind the spermatozoa determinative of one sex. The binding agent molecules are attached to magnetic particles. The methods also include applying an external magnetic field to the container to separate the magnetic particles from the remainder of the biological fluid sample containing spermatozoa determinative of the other sex and draining by gravity the remainder of the biological fluid sample from the container to separate the spermatozoa determinative of one sex from the remainder of the biological fluid sample containing the spermatozoa determinative of the other sex. The spermatozoa determinative of the other sex are then administered to the reproductive tract of a female mammal. The spermatozoa determinative of the other sex optionally are washed prior to administering the spermatozoa to the reproductive tract of a female mammal. In certain embodiments, the step of administering is artificial insemination. Preferably the mammal is one of cattle, sheep, pigs, goats, horses, dogs or cats, although other mammals can be the subject of the methods, including primates and exotic species.

In certain preferred embodiments of the methods, a “high dose” of spermatozoa is administered to the female mammal. In such embodiments, the number of spermatozoa administered is at least about 10 million, preferably at least about 20 million, more preferably at least about 30 million, more preferably at least about 40 million and still more preferably is at least about 50 million.

In certain preferred embodiments of the methods, a “low dose” of spermatozoa is administered to the female mammal. In such embodiments, the number of spermatozoa administered is less than about 10 million, preferably less than about 1 million, and more preferably less than about 0.5 million.

In some of these methods, the biological sample contains greater than about 1×10⁵ cells/ml.

In some of the foregoing methods, the binding agent molecules that selectively bind the spermatozoa determinative of one sex are antibodies. The selectivity of binding of the binding agent molecules may reflect differential expression of the antigen on the spermatozoa (by amount of expression or by time of expression) or other properties that permit one to selectively bind spermatozoa determinative of one sex. Thus, in some embodiments, the antibodies are specific for Y-bearing sperm or are specific for X-bearing sperm. In a preferred embodiment, the antibodies are specific for an H—Y antigen. Preferably the antibodies are monoclonal antibodies. Other selective binding agent molecules, such as lectins, phage display binding molecules and binding partners of a molecule on the spermatozoa determinative of one sex, also can be used.

The magnetic particle used in these methods preferably is a non-porous magnetic bead support, preferably having a diameter of 0.1 to 2 microns, more preferably having a diameter of 0.1 to 0.5 microns. In certain of the foregoing methods, the magnetic particle is covalently attached to the binding agent molecule.

According to still another aspect of the invention, methods for fractionating an entire ejaculate of a mammal in a single process are provided. The methods include obtaining an ejaculate, subjecting the ejaculate to the foregoing fractionation methods. In preferred methods, the ejaculate is fractionated with an efficiency of at least about 55%, at least about 56%, at least about 57%, at least about 58%, at least about 60%, at least about 65%, at least about 70%, at least about 75%, at least about 80%, at least about 85%, at least about 90%, at least about 95% or at least about 99%.

Methods of insemination are provided in another aspect of the invention. The methods include obtaining a mammalian ejaculate, fractionating the ejaculate according to the foregoing methods to obtain a population of spermatozoa, and inseminating a mammal with the population of spermatozoa. In preferred embodiments, the conception rate of offspring resulting from the insemination is at least about 50% of the conception rate obtained using unfractionated spermatozoa. More preferably, the conception rate is at least about 70%, still more preferably is at least about 80%, yet more preferably is at least about 90%, and most preferably is at least about 95% of the conception rate obtained using unfractionated spermatozoa.

In a further aspect of the invention, methods for creating a sex bias in mammalian offspring are provided. The methods include obtaining a population of spermatozoa from an ejaculate fractionated according to the methods disclosed herein and inseminating a mammal with the population of spermatozoa. In preferred methods, the ejaculate is fractionated in less than about 2 hours, more preferably in less than about 1 hour.

According to another aspect of the invention, methods for fractionating spermatozoa of a mammal without a substantial loss of motility are provided. The methods include obtaining an ejaculate containing spermatozoa, and subjecting the ejaculate to the methods of fractionation disclosed herein. In certain embodiments, the motility of the fractionated spermatozoa is at least about 50% of the unprocessed spermatozoa. Preferably the motility of the fractionated spermatozoa is at least about 60% of the unprocessed spermatozoa, more preferably is at least about 70% of the unprocessed spermatozoa, more preferably is at least about 80% of the unprocessed spermatozoa, more preferably is at least about 90% of the unprocessed spermatozoa, still more preferably is at least about 95% of the unprocessed spermatozoa, yet more preferably is at least about 97% of the unprocessed spermatozoa, more preferably still is at least about 98% of the unprocessed spermatozoa, and most preferably is at least about 99% of the unprocessed spermatozoa.

By using the methods of the invention disclosed herein, one can obtain fractionated populations of spermatozoa that have functionality comparable to unfractionated spermatozoa. Thus, in another aspect of the invention, populations of fractionated spermatozoa determinative of one sex are provided. In the populations of spermatozoa determinative of one sex, at least about 50% of the spermatozoa are motile. Preferably, at least about 60% of the spermatozoa are motile, more preferably at least about 70% of the spermatozoa are motile, more preferably at least about 80% of the spermatozoa are motile, more preferably at least about 85% of the spermatozoa are motile, more preferably at least about 90% of the spermatozoa are motile, still more preferably at least about 95% of the spermatozoa are motile, yet more preferably at least about 97% of the spermatozoa are motile, more preferably still at least about 98% of the spermatozoa are motile, and most preferably at least about 99% of the spermatozoa are motile.

It also has been discovered that there is in at least some instances a window of time following collection of ejaculates in which ejaculates can be more effectively fractionated into spermatozoa determinative of one sex. Therefore, in a further aspect of the invention, methods for fractionating an ejaculate of a mammal, are provided that include obtaining an ejaculate, and fractionating the ejaculate between about 2 hours and about 24 hours after collection of the ejaculate.

In preferred embodiments, the fractionation is carried out between about 2 hours and about 12 hours after collection of the ejaculate. More preferably, the fractionation is carried out between about 4 hours and about 8 hours after collection of the ejaculate. Still more preferably, the fractionation is carried out at about 6 hours after collection of the ejaculate.

It also has been discovered that in at least some instances the storage temperature of ejaculates can result in more effective fractionation of the ejaculate into spermatozoa determinative of one sex. Therefore, in a further aspect of the invention, methods for fractionating an ejaculate of a mammal are provided. The methods include obtaining an ejaculate, and fractionating the ejaculate after storage of the ejaculate at less than about 20° C. Preferably the ejaculate is stored at less than about 16° C., more preferably at less than about 12° C., still more preferably at less than about 8° C. and yet more preferably at less than about 4° C.

These and other embodiments of the invention are described in greater detail below.

### BRIEF DESCRIPTION OF THE DRAWINGS

The objects, advantages and features of aspects of the invention will be more clearly appreciated from the following detailed description, when taken in conjunction with the accompanying drawings, wherein like numbers are used for like features, in which:

FIG. 1 is a perspective view of a magnetic separator according to one illustrative embodiment of the invention;

FIG. 2 is a front view of the magnetic separator of FIG. 1 illustrated with a container for holding a test specimen and a vessel provided under the container for receiving a separated portion of the test specimen;

FIG. 3 is a cross-sectional view of the magnetic separator taken along section line 3-3 of FIG. 1;

FIGS. 4A-4D are schematic views illustrating a separation process of a test sample according to one illustrative embodiment of the invention;

FIG. 5 is a perspective view of a magnet assembly for the magnetic separator of FIG. 1;

FIG. 6 is an end view of the magnet assembly of FIG. 5; and

### DETAILED DESCRIPTION OF THE INVENTION

The present invention is directed to a magnetic separator for magnetically separating different components of a test sample. Generally, the test sample may be prepared as a mixture of magnetic components and non-magnetic components. A magnetic component can be affected, such as manipulated or controlled, within the mixture by the application of a magnetic field. It may be desirable to separate the mixed components from one another to allow a user to select one or both of the components for a desired property or properties.

The magnetic separator may have particular applications for separating cells, including purifying or isolating cells, according to cell surface properties of the cells, and a preferred application is for separating spermatozoa based on sex determinative factors. For example, the magnetic components may be substantially determinative of spermatozoa of one sex and the non-magnetic components may be substantially determinative of spermatozoa of the other sex such that a user may select either one of the separated spermatozoa pools for use in artificial insemination of mammals in an effort to produce offspring of a desired sex.

The magnetic separator employs a magnetic source or generator, hereinafter referred to as a “magnet”, that produces a magnetic field to separate the magnetic components from the non-magnetic components. The magnet may be configured to control movement of the magnetic components toward a desired region of the separator and away from the non-magnetic components. Once separated, the non-magnetic components may be removed from the magnetic separator substantially separate from the magnetic components with minimal remixing of the non-magnetic and magnetic components.

The magnetic separator may include a container-receiving region that is configured to receive a container for holding the test sample. The magnet may be arranged in the separator to produce an external magnetic field within the container-receiving region of the separator so as to act upon the test sample in the container. A guide may be provided to position the container at a predetermined location within the container-receiving region relative to the magnet to subject each test sample to a consistent magnetic field. One or more retainers may be provided to hold the container in a desired position relative to the magnet.

The separator may include one or more magnets that are positioned at predetermined locations about the container-receiving region to produce a desired magnetic field. For example, the separator may utilize a dipole arrangement in which a pair of magnets are positioned on opposite sides of the container-receiving region approximately 180° apart. However, any suitable magnet arrangement, such as three or four (quadripole) equally spaced magnets or multiple non-equally spaced magnets, may be incorporated in the separator. Each magnet may be a bar magnet formed from any suitable magnetic material. It is also contemplated that other suitable magnetic sources or generators, such as an electromagnet, may be utilized for the magnet.

The magnetic separator may include a container that is configured to hold the test sample within the magnetic field during separation, and then allow the non-magnetic components to be drawn off once separated. In this regard, the container may have an inlet for receiving a test sample and an outlet through which the non-magnetic components may be released after separation. The outlet may be positioned at a lower portion of the container to allow gravitational flow from the container. However, the inlet and outlet may be placed at any suitable location on the container. Additionally, the non-magnetic components may be removed from the container using any suitable device, such as a pump.

A regulator may be coupled to the outlet of the container to regulate the flow of the non-magnetic particles from the container. The regulator may be any suitable device to regulate the flow from the outlet, including a clamp or a valve, such as a stopcock.

The magnetic separator may be particularly suitable for separating a test sample having relatively high concentrations of magnetic and non-magnetic components. In this regard, the test sample may be held within the magnetic field by the container for a sufficient period of time to allow separation of the components. Once separated, the regulator may be actuated to release the non-magnetic components from the container at a controlled rate that reduces the likelihood that magnetic components would become remixed and drawn from the container along with the non-magnetic components. By controlling the movement of the magnetic components relative to the non-magnetic components, the magnetic separator may minimize or avoid remixing of the magnetic and non-magnetic components as compared to separators that rely on preventing movement of the magnetic components for separation.

The magnetic separator may include a base that supports the container-receiving region above a vessel-receiving region that is configured to receive a vessel for capturing the non-magnetic components released from the container.

To ensure adequate separation of the test sample has occurred prior to release of the non-magnetic components, the container may include a window or be formed of a transparent material to allow a user to visually monitor the amount of separation. Once the test sample is separated, the user may also monitor the test sample as the non-magnetic components are being released from the container to reduce the likelihood that magnetic components are inadvertently released from the container along with the non-magnetic components.

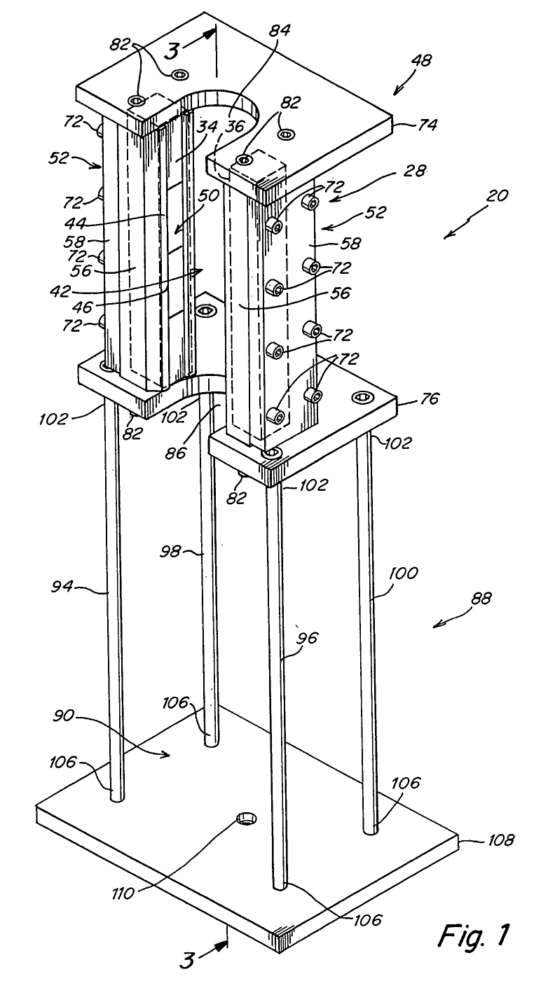

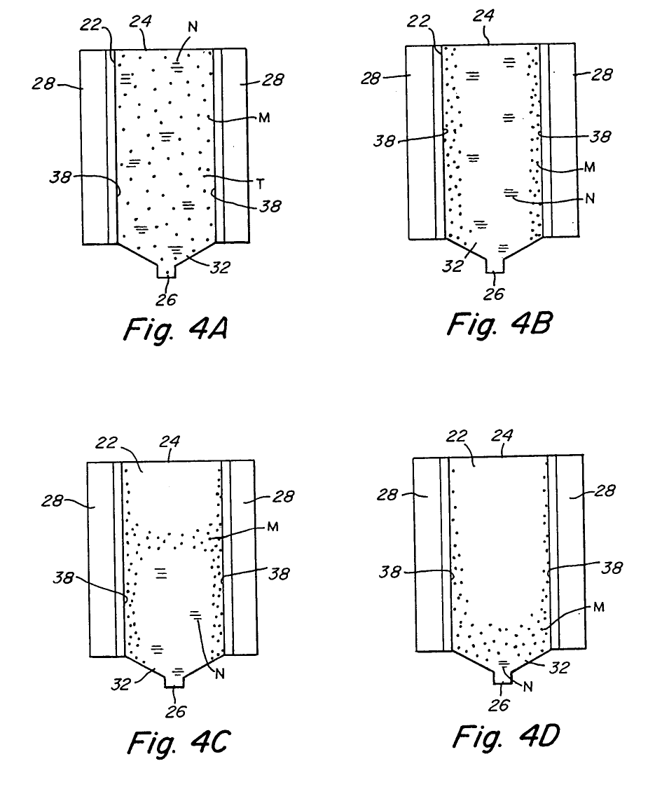

In one illustrative embodiment shown in FIGS. 1-3, the magnetic separator 20 includes a container-receiving region 42 that is configured to receive a container 22 for receiving and holding a test sample T (see FIG. 4A) and a magnet 28 to generate a magnetic field within the container 22 that will be operative on magnetic components within the test sample to substantially separate the magnetic components M from non-magnetic components N of the sample. The container 22 includes an inlet 24 for receiving the test sample and an outlet 26 through which the separated non-magnetic components may be released.

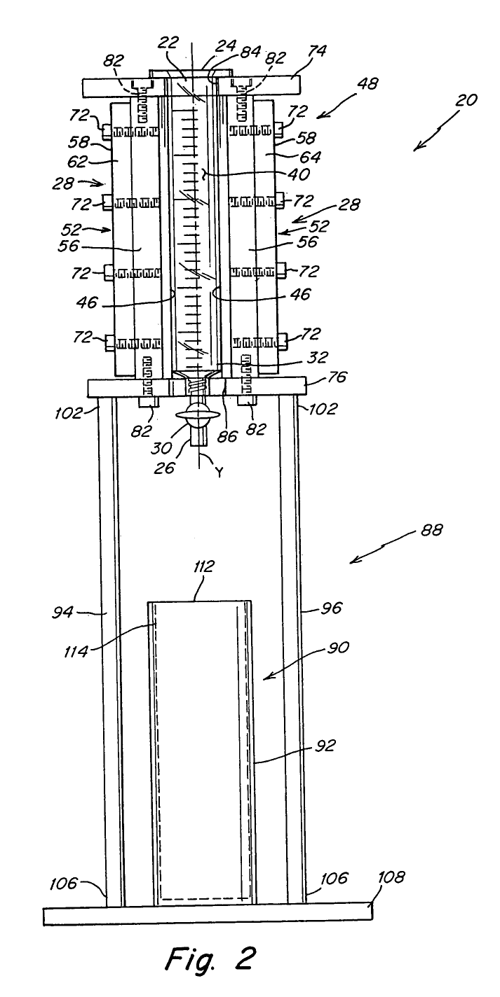

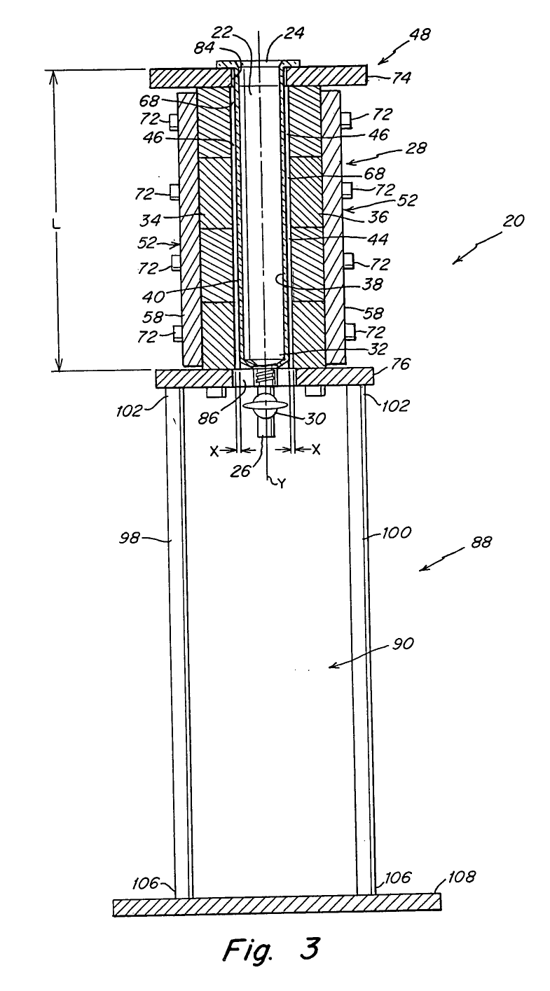

As shown in FIGS. 2 and 3, the inlet 24 is provided at the top of the container with the outlet 26 located below the inlet to allow gravitational flow of the non-magnetic components from the outlet. To maximize the amount of non-magnetic components that may flow from the container, the outlet is provided at the bottom 32 of the container. However, it is to be understood that the inlet 24 and outlet 26 may be provided at any suitable location on the container and have any suitable size or shape as would be apparent to one of skill in the art. In the illustrated embodiment, the bottom of the container has a substantially conical shape to facilitate the flow out of the container. However, it is to be appreciated that the bottom of the container may have any suitable shape, including a flat shape.

It may be desirable to regulate the flow of the non-magnetic components from the container to reduce the likelihood that magnetic components may inadvertently be released from the container. In one illustrative embodiment shown in FIGS. 2 and 3, a regulator 30 is coupled to the outlet 26 to regulate flow from the outlet of the container. As illustrated, the regulator 30 includes a stopcock that is attached to the outlet using a luer lock connection. However, it is to be understood that any suitable regulator, including a clamp provided to temporarily close the outlet, or a valve, may be utilized to control the flow of non-magnetic components from the container. Additionally, the regulator may be connected to the outlet of the container by any suitable connection.

The regulator may be actuatable between at least a closed position and an open position, such that the regulator may be used to stop or start flow from the outlet. The regulator may also be actuatable to one or more intermediate positions to vary the rate of flow from the outlet; for example, the rate of flow may be slowed or increased. Using the regulator, the flow from the outlet may be stopped for a sufficient period of time to allow the magnetic and non-magnetic components to substantially separate from one another in the container. This arrangement may be particularly suitable for test samples having high concentrations of magnetic components. Also, the flow of the non-magnetic components from the outlet may be regulated, such that the flow of non-magnetic components stays substantially free of the magnetic components by reducing the likelihood of remixing caused by too fast a flow from the outlet. A user may use the regulator to stop the flow from the outlet, for example when substantially all of the non-magnetic components have flowed from the container and the user wants to stop the flow before the magnetic particles flow from the outlet. Although use of a regulator may provide certain advantages, it is to be appreciated that other embodiments of the magnetic separator may not include a regulator 30 at the outlet 26 of the container 22.

In one embodiment, the container 22 includes a standard 30 ml syringe barrel. However, the magnetic separator may be configured to accommodate any syringe barrels of any desired size, for example 20, 40, 50 or 60 ml syringe barrels. It is also to be appreciated that the magnetic separator may be configured to utilize a container having any suitable size or shape.

To ensure that adequate separation of the test sample has occurred, at least a portion of the container 22 may be substantially transparent, such that the test sample within the container may be visible from outside the container. For example, the entire container may be formed from a transparent material, or a portion of the container may be transparent, such as by having a window in the container through which to view at least a portion of the test sample. The transparency of at least a portion of the container may allow a user to monitor the separation of the magnetic and non-magnetic components and to ensure that substantially only the non-magnetic components may be allowed to flow from the outlet. However, it is to be understood that the container need not be constructed to allow visibility of the test sample. For example, the container may be opaque or the magnets may obstruct the view of the test sample in the container. It will be appreciated that the container may be made of any suitable material, such as metals or plastics.

In certain instances it may be desirable to have a tube connected to the outlet 26 to provide visibility of the test sample as it exits the container. For example, a tube may be particularly useful when the magnets substantially block the view of the test sample in the container. The tube may be directly coupled to the outlet, or to a regulator.

Although the illustrative embodiment of the magnetic separator is configured as a gravity flow system, it will also be appreciated that a pump (not shown) may be provided to assist in releasing the non-magnetic components from the outlet. The pump may be any suitable pump apparent to one of skill in the art, including a peristaltic pump, a syringe and the like.

One illustrative embodiment of a process of separating the magnetic components from the non-magnetic components is shown in FIGS. 4A-4D. However, it is to be appreciated that the described process is merely exemplary and the magnetic separator may be employed to carry out other processes as would be apparent to one of skill in the art.

FIG. 4A illustrates a test sample T that includes a mixture of magnetic components M and non-magnetic components N placed within the container 22 of the separator. The magnet 28 generates a magnetic field within the container that is operable upon the magnetic components M within the test sample to substantially separate the magnetic components and non-magnetic components N.

When subjected to the magnetic field for a sufficient amount of time, the magnetic components are attracted and migrate toward an inner surface 38 of the container 22 as shown in FIG. 4B. For a test sample having a high concentration of magnetic components, multiple layers of magnetic components may be formed along the inner surface of the container during the separation process. During separation, the magnetic components may form a magnetic phase within the test sample that is separate from the non-magnetic components.

Once separated, the non-magnetic components N may be released through the outlet 26 of the container as shown in FIG. 4C and into a separate vessel configured to capture the components released through the outlet. As the non-magnetic components N are released from the container, a portion of the magnetic components M may become dislodged and gather together to form a bolus of magnetic components at a top portion of the test sample. Although it is generally desirable for the non-magnetic components to flow substantially separately from the container, it is to be appreciated that some minimal amount of magnetic components may remix and inadvertently flow from the outlet 26 along with the non-magnetic components.

As the amount of non-magnetic components in the container decreases, the bolus of magnetic components M moves closer to the outlet as shown in FIG. 4D. When substantially all the non-magnetic components have been released from the container, the user may close off the outlet 26 to reduce the likelihood of releasing magnetic components from the container. Once the non-magnetic components have been drained from the container, it may be desirable to flush the magnetic components out of the container and into another vessel separate from the non-magnetic components.

The magnet 28 may include any suitable magnetic source or generator that produces a magnetic field within the container. In one illustrative embodiment shown in FIGS. 1-3, the magnet includes two bar magnets 34 and 36 aligned parallel to one another on opposite sides of the container in a di-pole or multi-pole arrangement for a magnetic separator. Each bar magnet has a polarity including a North (N) and South (S) pole. The bar magnets may be oriented with the opposite poles of the bar magnets facing one another, such as North (N) facing South (S), causing the bar magnets to attract one another. Alternatively, bar magnets may be oriented with the same poles facing each other, such as North (N) facing North (N) or South (S) facing South (S), causing the bar magnets to repel one another. Each bar magnet may include a plurality of magnets that are longitudinally stacked with each other to form the bar magnet.

As illustrated, the bar magnets may be equally spaced from one another about the container. Although the magnet 28 is illustrated as having two bar magnets spaced approximately 180° apart about the container, it will be appreciated that the bar magnets may be located in any suitable position about the container. Additionally, although two bar magnets are shown, any number less than or greater than two may be employed.

In one illustrative embodiment shown in FIG. 3, the bar magnets are spaced by a distance X from the container. This spacing may be desirable to allow the container to be more readily placed within or removed from the container-receiving region. However, it will be appreciated that the bar magnets may directly contact an outer surface 40 of the container.

In the illustrative embodiment, the bar magnets extend in a direction that is substantially aligned with a longitudinal axis Y of the container. As shown, each of the bar magnets has a length L that is substantially equal in length to the container to produce a magnetic field substantially throughout the entire container. It is to be understood, however, that the magnetic separator may utilize a magnet having any suitable orientation, size or shape. For example, the magnet may surround at least a portion of the container about its longitudinal axis Y and extend along at least a portion of the length of the container. In this regard, the magnet may have an annular shape such that the magnet fits about the container and its longitudinal axis.

Although the magnet 28 is illustrated as including a pair of bar magnets 34 and 36, it is to be appreciated that other arrangements are contemplated. In another embodiment four bar magnets may be equally spaced about the container in a quadri-pole arrangement. It is to be understood that if more than one magnet is provided, the magnets may be equally spaced about the container, or may be randomly spaced about the container. Further, each magnet may be spaced from the container or directly contact the outer surface of the container.

The magnet 28 may be made of any suitable material apparent to one of skill in the art. For example, the magnet may be formed from one or more of niodymium iron boron, samarium cobalt, alnico or ferrite. The magnet may also include a flexible magnet, such as those made of ferrite in a vinyl carrier. Any suitable material for the magnet may be used to generate the magnetic field of a desired strength. Moreover, as would be apparent to one of skill in the art, pole pieces may be included to assist in generating the desired magnetic field within the container. As is also understood in the art, multiple magnets may be yoked, such that the magnets are connected to one another by a ferrous material to generate the desired magnetic field.

In another embodiment, the magnet 28 may include one or more electromagnets that may be selectively turned on to generate the magnetic field. For example, one or more electromagnets may be provided spaced from or in contact with the container. If more than one electromagnet is provided, they may be equally or randomly spaced about the container, as described above.

In some applications, it may be desirable to generate a closed magnetic field within the container. In one embodiment, a ferrous material, such as steel wool, may be provided within the container 22 that interacts with the magnet 28 to form a closed magnetic field. The ferrous material may be coated in a manner apparent to one of skill in the art to avoid direct contact between the biological components of the test sample and the ferrous material.

However, it is to understood that an uncoated ferrous material may be used, if desired. As described above, the magnet 28 is arranged to generate a magnetic field within the container-receiving region 42. As illustrated, at least one guide 46 may be provided to receive the container 22 in the container-receiving region. Also, at least one retainer 44 may be provided to hold the container adjacent the magnet. As illustrated in FIG. 3, the guides position the container in the container-receiving region at a substantially equal distance X from each magnet 34 and 36. The retainers and guides may have any suitable configuration and they may be formed unitarily or as separate pieces.

As illustrated in FIGS. 1-3, the separator may include a housing 48 that defines the container-receiving region 42 and maintains the magnet 28 adjacent the container-receiving region. The housing may provide the magnet spaced the distance X from the container-receiving region as described above. The housing may include the guide 46 or retainer 44 to receive a portion of the outer surface 40 of the container and to hold the container adjacent the magnet. In the illustrative embodiment, the housing includes a pair of guides 46 that are located on opposite sides of the container-receiving region to receive and hold the container in the container-receiving region. The guides 46 define a channel 50 that receives at least a portion of the outer surface of the container. The guides may slidably receive the container, for example from the top of the housing by sliding the container into the container-receiving region from above, or may receive the container by a snap-fit configuration, such as by inserting the container into the container-receiving region from the side.

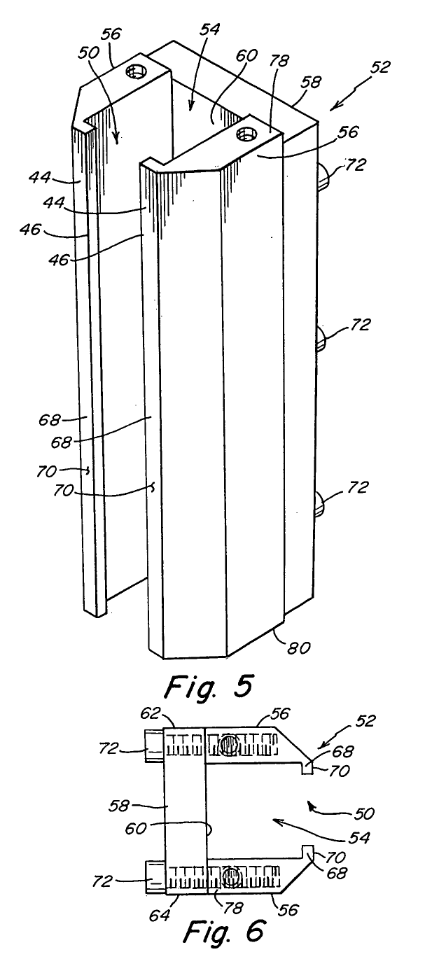

The housing 48 may include a magnet assembly 52 that is configured to hold and maintain the bar magnets adjacent the container-receiving region. In one illustrative embodiment shown in FIGS. 5 and 6, each magnet assembly includes one or more magnets to form the bar magnets 34 and 36. The magnet assemblies are shown substantially equally spaced from one another about the container-receiving region, and therefore, about the container when placed within the container-receiving region. It will be appreciated, however, that they may be unequally spaced about the container.

In the illustrative embodiment shown in FIGS. 5 and 6, each magnet assembly 52 includes a receptacle 54 to receive the magnet or magnets. Each magnet assembly includes a pair of magnet holders 56 that extend from a magnet cover 58. Each magnet holder is secured to a surface 60 of the magnet cover and adjacent its opposite edges 62 and 64. The magnet holders are substantially parallel and spaced from one another to form the receptacle 54 for receiving the magnet. As shown, the magnet assembly is constructed of separate pieces secured together using any suitable fasteners 72, such as screws. However, the magnet assembly may be formed as a unitary piece, or may be formed of multiple pieces and secured together in any suitable manner.

As illustrated, each magnet holder has a lip 68 that is spaced from the magnet cover for securely holding the magnet within the receptacle 54 of the magnet assembly. An outer surface 70 of the lip 68 is configured to form the guide 46 and/or retainer 44 to receive and hold at least a portion of the outer surface 40 of the container. For example, the outer surface 70 of each lip of the magnet assembly, along with the channel 50 provided therebetween, may receive at least a portion of the outer surface of the container.

Although a particular embodiment of the magnet assembly 52 is shown and described, it will be appreciated that the magnet assembly may be any suitable shape or configuration to receive the magnets and/or act as a guide to receive and retain the container within the container-receiving region.

As illustrated in FIGS. 1-3, the housing 48 includes a top plate 74 and a bottom plate 76 secured to opposite ends 78 and 80 of the magnet assemblies 52 using any suitable fasteners 82, such as screws. It will be appreciated that the plates may be secured to the magnet assemblies by any other suitable means, including welds or adhesive. The plates act to secure the magnets within the magnet assemblies by blocking the ends of the magnet assemblies. Although a particular embodiment of the top and bottom plates of the housing are illustrated and described, they may have any suitable configuration.

As illustrated, each plate has an opening 84 and 86 for accommodating the container. For example, the container may be inserted through the top opening 84 in the top plate, and received by the guides 46. The bottom of the container may extend through the bottom opening 86 in the bottom plate such that the outlet 26 is not blocked. Alternatively, the container may be inserted into the retainers and opening from the side, for example by a snap-fit, such that the container extends above the top plate and below the bottom plate by being within the top and bottom openings. The openings are shown as having an open side; however, the openings may have any suitable shape, such as a substantially circular shape with no open sides.

The various parts of the housing 48 may be made of any suitable material including metal, such as aluminum, or a plastic material. Further, the components of the housing may be made of different materials. Moreover, the housing may be formed as a unitary piece or of multiple pieces suitably secured together.

As illustrated in FIGS. 1-3, the magnetic separator includes a base 88 for supporting the container-receiving region 42. In one illustrative embodiment, the base has a vessel-receiving region 90 for receiving a vessel 92 (FIG. 2) below the container-receiving region to capture the non-magnetic components that flow from the outlet 26 of the container 22. Although a particular embodiment of the base 88 is shown and described, the base may be any suitable configuration for providing a vessel-receiving region, such that a vessel may be placed in the region to receive the non-magnetic components as they flow from the container. For example, the base may be a solid structure with a hollowed-out opening to receive the vessel below the container-receiving region.

In the illustrative embodiment, the base 88 includes four upstanding legs 94, 96, 98 and 100 that are secured at their upper ends 102 to the bottom plate 104 of the housing 48 and at their lower ends 106 to a base plate 108. The legs elevate the container-receiving region 42 above the vessel-receiving region 90. As shown, the legs are substantially parallel to one another. It will be appreciated that the legs may be any suitable size or shape. For example, although the legs are shown having a small circular cross-section, they may have a rectangular cross-section and may be any suitable size. It will also be appreciated that although four legs are illustrated, one or more legs may be used to elevate and support the container-receiving region.

The base plate allows the magnetic separator to stand substantially freely on a surface. For additional support, if desired, the base plate may include an aperture 110 to secure the base 88 to a surface using a releasable fastener (not shown). It will be appreciated, however, that the base may be secured to a surface using any suitable means.

The base may be formed as a unitary structure or of multiple pieces suitably secured together. The base may be formed of any suitable materials, including various metals and plastics.

As shown in FIG. 2, the vessel 92 has an opening 112 at a top portion 114 for receiving the non-magnetic components that flow from the outlet 26 of the container 22. The vessel may be any container suitable to receive and hold the non-magnetic components that flow from the outlet of the container. The vessel-receiving region may be configured to allow the vessel to be readily placed within and removed from the vessel-receiving region for capturing the non-magnetic components. Moreover, it will be appreciated that the vessel may receive the non-magnetic components directly from the outlet or through some other device, such as the regulator or tubing.

The invention also provides methods for magnetically separating a selected population of cells from a biological sample using the device described above. A biological sample is a sample that contains some biological component, in this case cells that are to be separated. The methods are particularly useful for separating large populations of cells without the application of excessive force (e.g., centrifugal force) or harsh environments (e.g., chemicals, contact with objects such as steel wool) that can be destructive to cells and/or can impair biological properties of cells. In preferred embodiments, cells are separated based on properties of their surfaces, e.g., protein, lipid or carbohydrate molecules that are on the surface of the cells or project from the surface of the cells.

In operation, the methods includes the step of contacting the biological sample in a container with a plurality of binding agent molecules that selectively bind the selected population of cells, for a time sufficient for the binding agent molecules to bind the cells, to form a magnetic component of the biological sample. An external magnetic field is then applied to the container (i.e., using the magnetic separation device) to separate the magnetic component from the non-magnetic components of the biological sample. The non-magnetic components of the biological sample then are removed from the container to separate the selected population of cells from the non-magnetic components of the biological fluid sample. Typically the removal of the non-magnetic components from the container is by draining the non-magnetic components out of the bottom of the container.

In alternative embodiments, a reaction mixture is formed by contacting the biological fluid sample with a binding agent that selectively binds the selected population of cells for a time sufficient for the binding agent to bind the selected population of cells. The reaction mixture then is transferred to a separation container in which an external magnetic field is applied to separate the magnetic particles from the biological fluid sample. The non-magnetic components of the biological sample are then removed from the container to separate the selected population of cells from the non-magnetic components of the biological fluid sample.

The binding agent molecule can be unlinked to a magnetic particle or linked to a magnetic particle when added to the biological sample. When unlinked binding agents are used, the binding agent is contacted with the biological sample for a time sufficient to bind the selected population of cells. A magnetic particle containing a linking compound is subsequently added to link the binding agent molecule to the magnetic particle. The linking of binding agent molecules to magnetic particles is described further below.

Typically the removal of the non-magnetic components from the container is performed by draining the non-magnetic components out of the bottom of the container. The importance of draining the non-magnetic components from the container, rather than removing these components by aspiration, is that aspiration tends to mix the liquid by creating vortices and other turbulent fluid movement. In methods such as cell separation, particularly as with the methods of the invention in which the magnetic field applied does not necessarily hold the magnetized component immobile against container walls, it is important to keep turbulent fluid movement to a minimum. This preferably is achieved by removing fluid from the bottom of the container, using laminar flow, which limits remixing of the sample. Thus, in a preferred embodiment, the step of draining the selected population of cells from the container is performed by draining the container by gravity. Other methods for draining the container, such as by pump or regulated pressure also can be used. In a typical application, the step of draining is regulated by opening and optionally closing a valve or stopcock to regulate the flow of the non-magnetic components from the container. If a pump is used, then regulating the operation of the pump attached to a drain of the separator container will achieve the same effect.

Another method for removing the selected population of cells from the container without disturbing the cell separation includes pumping a dense fluid (i.e., denser than the non-magnetic components to be removed) into the container to displace the non-magnetic components of the biological sample from the container. Using this method, the non-magnetic components can be removed from the top of the container rather than by draining from the bottom.

A wide variety of cells can be separated by the methods described herein. As used herein, cells includes eukaryotic cells (including mammalian cells, nucleated cells, enucleated cells, etc.), cell fragments, prokaryotic cells, virus particles, etc. A preferred population of cells for separation into subpopulations is spermatozoa, in which spermatozoa determinative of one sex are desired.

In certain uses, the biological sample will include a second population of cells that is not recognized and bound by the binding agent molecules. Because these non-recognized cells will not be bound by binding agent and thus will not be physically associated with magnetic particles used in the separation process, the second population of cells (non-recognized cells) are non-magnetic components of the biological fluid sample.

One of the features of the use of the magnetic separator is that separated populations of cells can be recovered from the device after separation with little waste. Using spermatozoa as an example (but equally applicable to other populations of cells, such as lymphocytes), one can fractionate the cells using monoclonal antibodies specific for Y-bearing spermatozoa attached to magnetic particles. In this example, the magnetic separator “pulls” out the Y-bearing sperm recognized by the antibodies, and X-bearing sperm (now the non-magnetic component of the biological sample) can be drained out of the magnetic separator. The magnetic separator is constructed to permit all but a small amount of the non-magnetic component to be removed from the separation container; the small amount is left behind to ensure that only the desired population of cells is recovered. In operation this is similar to removing the bottom phase in a separatory funnel. After removing the non-magnetic components, the small amount of non-magnetic components is removed (similar to the interface between phases in a separatory funnel). This leaves the magnetic components in the separation container, i.e., the cells that are bound to the binding agent (e.g., antibodies). These cells can also be recovered, thus providing separation and isolation of the two populations (X- and Y-bearing) of spermatozoa. Recovery of the magnetic component is easily performed by removing the separation container from the magnetic separator and then draining the container.

The separation methods using the magnetic separator can be repeated sequentially on a single population of cells to further purify the cells (e.g., using the same or a different binding agent that also recognizes the cells or a subpopulation thereof), or can be repeated sequentially on a mixed population of cells using binding agent molecules that bind to different populations of cells in order to recover several different populations of cells.

In routine operation of the magnetic separator-device to separate large populations of cells, the magnetic field is insufficient to hold the magnetic particles to the surface of the container, i.e., proximal to the externally applied magnetic field. In certain instances, due to the number of cells and magnetic particles bound to the cells, the magnetic particles are too numerous to form a monolayer of particles on the walls of the container under the influence of the magnetic field, i.e., the number of particles is too great for surface area. This contrasts with other magnetic separation devices that rely on higher field strength to hold the magnetic particles to the walls of the separator.

In some instances, the magnetic component of a sample (e.g., magnetic beads and bound cells) and the non-magnetic component of a sample can form distinct and separate phases that are conveniently separated by removing one of the phases from the separation container. The separator device facilitates this removal by providing a drain that can regulate the outflow of the non-magnetic components of the biological sample as described above. Although not wishing to be bound to any particular theory, it is believed that the phase separation may be due to the differences in the induced viscosity of the magnetic component phase of the sample and the non-magnetic component phase of the sample when exposed to the magnetic field in the separator device, with the magnetic components restricted to a smaller volume (and thus having a high induced viscosity). Under certain conditions, the selected population of cells that is bound to the magnetic particles can form a “bolus” that protrudes from the interior surface of the container. This is most frequently seen upon draining of the non-magnetic components of the sample. In certain instances, the bolus can extend from the sides sufficiently to meet in the middle of the separation container, but remains distinct and separated from the non-magnetic components.

Particular binding agent molecules that are useful for separating a desired population of cells due to their cell recognition properties will be known to one of ordinary skill in the art. The binding agent molecules can be of any kind that bind cells with sufficient affinity and/or avidity to remain bound during the separation procedures. Exemplary binding agent molecules include antibodies, lectin molecules, phage display molecules (or other combinatorial binding molecules), binding partners of a cell surface molecule (e.g., one of a ligand-receptor pair such as CD4-CD4 receptor; a carbohydrate or carbohydrate-containing molecule (such as a glycoprotein) and a carbohydrate receptor on the cell surface), etc.

In some preferred embodiments, the binding agent molecule used in the methods is an antibody or antigen-binding fragment thereof. Particular antibodies and other binding agent molecules will be preferred for their ability to distinguish between closely related populations of cells. For example, to separate spermatozoa, antibodies that bind cell surface molecules that permit one to distinguish Y-bearing spermatozoa from X-bearing spermatozoa will be useful. These antibodies bind cell surface molecules that differ in type or crypticity or other properties between X-bearing spermatozoa and Y-bearing spermatozoa. A variety of these antibodies are known to one of ordinary skill in the art, such as the antibodies that recognize H—Y antigen used in the Examples (see U.S. Pat. No. 4,680,258 to Hammerling et al.), or the sex-specific antibodies that bind to sex-chromosome-specific proteins on the sperm membrane described by Blecher et al. (Theriogenology 52(8):1309-1321, 1999; U.S. Pat. No. 5,840,504).

The invention, therefore, embraces peptide binding agents which, for example, can be antibodies or fragments of antibodies having the ability to selectively bind to polypeptides, carbohydrates or other cell-surface molecules. Antibodies include polyclonal and monoclonal antibodies, prepared according to conventional methodology. Monoclonal antibodies are preferred for use in the methods described herein.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fe regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. See, e.g., U.S. Pat. Nos. 5,545,806, 6,150,584, and references cited therein. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences.

Thus, as will be apparent to one of ordinary skill in the art, the present invention also provides for the use in separation methods of F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes the use of so-called single chain antibodies.

Accordingly, the invention involves polypeptides of numerous size and type that bind specifically to cell-surface molecules, including polypeptides, carbohydrates, lipids, and combinations thereof. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to a particular cell type that is to be separated using the magnetic separator device. This process can be repeated through several cycles of reselection of phage that bind to the particular cell type. Repeated rounds lead to enrichment of phage bearing particular sequences. DNA sequence analysis can be conducted to identify the sequences of the expressed polypeptides. The minimal linear portion of the sequence that binds to the particular cell type can be determined. One can repeat the procedure using a biased library containing inserts containing part or all of the minimal linear portion plus one or more additional degenerate residues upstream or downstream thereof.

Yeast two-hybrid screening methods also may be used to identify polypeptides that bind to the particular cell type.

Antibodies and other binding agent molecules are bound to the magnetic particles (also referred to herein as magnetic beads) using procedures which are well known to the person of ordinary skill in the art. Antibodies and other binding agent molecules can be covalently linked directly to the magnetic particles, or can be attached to the magnetic particles through an intermediate linking compound. In general, a linking compound is attached to the magnetic beads during manufacture of the beads. An antibody then is bound by the linking compound on the beads, for example by mixing beads at about 1 mg iron/ml with purified antibody at 1 mg/ml protein. After the antibody is bound to the beads, the beads are washed so only attached antibody remains. Additional procedures known to those skilled in the art are described, for example, in U.S. Pat. No. 4,018,886; U.S. Pat. No. 3,970,518; U.S. Pat. No. 4,855,045; and U.S. Pat. No. 4,230,685.

Examples of an intermediate linking compound for antibodies include Protein A, Protein G, and other proteins that specifically bind antibodies, lectins, receptors and the like, including antibodies that bind other antibodies, such as anti-Fc antibodies, anti-IgG antibodies or anti-IgM antibodies. Protein A is a preferred linking compound which greatly increases the effectiveness of capture by the attached antibodies. (Forsgren et al., (1977) J. Immunol. 99:19). Protein A attaches to the Fe portion of IgG subclass antibodies, thus extending and presenting the Fab portion of these antibodies. The resulting correct orientation of the antibodies and extension away from the magnetic particles leads to a very effective interaction between the bound antibodies and their target.

The method of attachment of Protein A to magnetic particles may proceed by any of several processes available to one of ordinary skill in the art. In one such procedure, magnetic iron oxide particles of approximately one micrometer diameter are chemically derivatized by a reaction, first with 3-aminopropyltriethoxysilane, then with glutaraldehyde. The derivatized magnetic particles are then mixed with Protein A resulting in a magnetic particle to which Protein A is covalently attached. The antibodies are then added to the Protein A magnetic particles and after a short incubation, the Protein A-antibody complexes form (Weetall, H. H. (1976) Meth. Enzymol. 44:134-48).

Magnetic particles preferably are non-porous magnetic beads. Preferably the diameter of the beads is less than about 10 microns, more preferably less than about 5 microns. The particular bead magnetic particles that provide an optimal recovery of a desired population of cells can be selected by one of ordinary skill in the art by testing particles of different sizes and properties using the magnetic separator describe herein to carry out the methods of the invention. In particularly preferred embodiments, the magnetic beads have a diameter of 0.1 to 2 microns, and more preferably have a diameter of 0.1 to 0.5 microns. Additional useful magnetic beads are described, for example, in U.S. Pat. No. 5,071,076; U.S. Pat. No. 5,108,933; U.S. Pat. No. 4,795,698; and PCT Publication No. WO91/09678.

As noted above, the methods for cell separation using the magnetic device of the invention are particularly useful for separating large populations of cells. In these embodiments, the biological sample contains greater than about 1×10⁵ cells, greater than about 1×10⁶ cells, greater than about 1×10⁷ cells, greater than about 1×10⁸ cells, greater than about 1×10⁹ cells, or more. The separator device differs from other cell separation devices in several ways, including that it permits the rapid and gentle separation of large quantities of cells. In contrast, well known methods such as fluorescence activated cell sorting cannot separate large numbers of cells in a single run, but rather take a long time and subject cells to harsh conditions. In certain embodiments, the number of cells in the selected population of cells separated using the separator device is greater than about 1×10⁵ cells/ml. Larger populations of cells are readily separated, such as populations of greater than about 5×10⁵ cells/ml, greater than about 1×10⁶ cells/ml, greater than about 2×10⁶ cells/ml, greater than about 1×10⁷ cells/ml and greater than about 1×10⁸ cells/ml.

The ability to separate efficiently and quickly a large number of cells permits the separation of cells for artificial insemination applications, particularly for agricultural uses in which multiple ejaculates must be separated to service large insemination operations. Thus, the separation of spermatozoa from animal ejaculates into spermatozoa determinative of one sex is a preferred use of the separator device. The ability to separate efficiently a large number of cells also permits the separation of whole ejaculates, without discarding any of the desired type of spermatozoa. Thus whole ejaculates can be used efficiently in contrast to existing methods in which portions of desired spermatozoa are discarded or wasted in the processing procedure.

The efficiency and gentleness of the cell separation using the magnetic separator of the invention provides opportunities for methods of artificial insemination in which a population of spermatozoa obtained using the magnetic separator is used to inseminate a mammal. Standard methods of artificial insemination that are well known in the art can be used, including combining separated spermatozoa with standard extension composition (e.g., including egg yolk and various other components), packing separated spermatozoa into straws and optionally storing them, and inseminating animals with the separated spermatozoa. It is even possible to use the magnetic component of the separation methods without further purification from the magnetic particles, i.e., a selected population of cells that are bound to magnetic particles.

Therefore the separator device and methods of using it provide for methods of increasing the percentage of mammalian offspring of either sex. The methods include magnetically separating spermatozoa determinative of one sex from a biological sample containing spermatozoa determinative of both sexes by carrying out the methods described herein for use of the magnetic separator device. Once the spermatozoa determinative of one sex are separated from the remainder of the biological fluid sample containing the spermatozoa determinative of the other sex, either population of separated spermatozoa can be administered to the reproductive tract of a female animal, preferably a mammal, preferably using artificial insemination techniques. Further steps, such as washing the isolated and separated spermatozoa prior to administering the spermatozoa to the reproductive tract of a female animal also can be performed. As used herein, “mammal” includes cattle, sheep, pigs, goats, horses, dogs, cats, primates or other mammals.

Artificial insemination techniques can use either “high dose” or “low dose” methods (reflecting the relative amounts of spermatozoa used for insemination; the methods of the invention are applicable with any amount of spermatozoa (i.e., including both high dose and low dose methods). In certain embodiments of the methods using spermatozoa separated using the device of the invention, a relatively high dose is used, e.g., greater than about 10 million cells are used for insemination. In these embodiments, the number of spermatozoa administered preferably is at least about 20 million, more preferably at least about 30 million, still more preferably at least about 40 million, and yet more preferably at least about 50 million. In other embodiments of the methods using spermatozoa separated using the device of the invention, a relatively low dose is used, e.g., less than about 10 million cells are used for insemination. In these latter embodiments, the number of spermatozoa administered preferably is less than about 5 million, more preferably is less than about 1 million and still more preferably is less than about 0.5 million.

The use of the magnetic separator, because it can efficiently and gently separate large numbers of cells with low cell loss, also provides the ability to fractionate an entire ejaculate of a mammal in a single process, which is not achievable using current methods of cell separation such as fluorescence activated cell sorting (FACS). FACS typically loses greater than 90% of the input cells. The ability of the methods of the invention to separate large numbers of cells with low cell loss is an advantage for artificial insemination operations and other organizations that process many ejaculates. The ability to fractionate entire ejaculates is advantageous even for smaller organizations and individual farmers that may separate spermatozoa from only their own herd.

To fractionate an entire ejaculate, it is combined with magnetic particles and binding agent molecules selective for spermatozoa determinative of one sex, preferably monoclonal antibodies, and then subjected to separation using the magnetic separator as described above. The ejaculate in some embodiments is fractionated with an efficiency of at least about 55%, although higher efficiencies of fractionation into populations of spermatozoa determinative of one sex is preferably performed with higher efficiency, such as at least about 56%, at least about 57%, at least about 58%, at least about 60%, at least about 65%, at least about 70%, at least about 75%, at least about 80%, at least about 85%, at least about 90%, at least about 95%, or at least about 99%. Other cell types can be separated with similar high efficiencies.

Subsequent to fractionating the ejaculate, animals (preferably mammals) can be inseminated with the population of spermatozoa determinative of one sex. Because the methods of fractionation and cell separation using the magnetic separator are efficient and gentle to cells that are easily damaged, such as spermatozoa, the cells isolated using the methods retain most if not all of their activity as compared to unfractionated cells. For spermatozoa, this means that conception rates for animals inseminated with fractionated cells are maintained at levels similar to that using unfractionated cells. In contrast, prior methods of cell separation often compromise the motility and fertilization ability of spermatozoa due to the use of harsh conditions including exposure to laser light and dye molecules (FACS), shear forces, etc., so that fertilization utilizing such separated spermatozoa requires complicated and expensive techniques and lowers the efficiency of conception. Thus, using the magnetic separator of the invention to separate spermatozoa that are then used in standard insemination procedures, the conception rate of offspring resulting from the insemination is, in preferred embodiments at least about 50% of the conception rate obtained using unfractionated spermatozoa. In more preferred embodiments, the conception rate is higher and approaches that seen using unfractionated spermatozoa (e.g., at least about 70%, 80%, 90%, or 95% of the conception rate obtained using unfractionated spermatozoa). These methods are, therefore, useful for creating a sex bias in mammalian offspring without the use of IVF, embryo transfer or other expensive procedures.

Another feature of the separation using the magnetic separator of the invention is its ability to quickly fractionate large numbers of cells, which is particularly useful for separation of cells where biological activity must be retained as much as possible. For example, an entire ejaculate can be fractionated in less than about 2 hours. Preferably an entire ejaculate is fractionated in less than about 1 hour. This can be contrasted with FACS methods that require many more hours to process large numbers of cells at low yield (e.g., about 7-10 straws per day), thereby exposing the cells to dye compounds for long times and long storage times while awaiting fractionation.

By using the magnetic separator in accordance with the methods described herein, spermatozoa of a mammal can be fractionated quickly and without a substantial loss of functionality. Functionality includes, but is not limited to: motility, progressive motility, acrosomal integrity, post-thaw motility and morphology. Thus, the functionality of the fractionated spermatozoa using these methods is at least about 50% of the unprocessed spermatozoa. Preferably the functionality of the fractionated spermatozoa is at least about 60% of the unprocessed spermatozoa, at least about 70% of the unprocessed spermatozoa, at least about 80% of the unprocessed spermatozoa, or is at least about 90% of the unprocessed spermatozoa. More preferably, the functionality of the fractionated spermatozoa is at least about 95% of the unprocessed spermatozoa, still more preferably is at least about 97% of the unprocessed spermatozoa, yet more preferably is at least about 98% of the unprocessed spermatozoa, and most preferably is at least about 99% of the unprocessed spermatozoa. Populations of fractionated spermatozoa determinative of one sex having the foregoing levels of functionality relative to unprocessed spermatozoa are also provided.

In another aspect of the invention, methods are provided for fractionating ejaculates based on an unexpected criticality of time and temperature in certain aspects of the separation process. It has been discovered that the time after collection of the ejaculate and the temperature at which the ejaculates are stored and/or handled are unexpectedly important for efficient separation of spermatozoa determinative of male and female offspring. It was determined that there is a “window” of time for efficient separation of these spermatozoa types. The window of time “opens” for efficient separation at about 2 hours after collection of an ejaculate, and “closes” by about 24 hours after collection of an ejaculate. This was shown by the ability of an antibody to bind preferentially to Y-chromosome bearing spermatozoa (e.g., with greater avidity than binding to X-chromosome bearing spermatozoa) within this time window, and also by the results of insemination of animals with spermatozoa separated either within the favored time window or outside of the favored time window. These results are reported in the Examples below. Thus the invention provides methods for fractionating an ejaculate (or separating spermatozoa determinative of male and female offspring) by fractionating the ejaculate between about 2 hours and about 24 hours after collection of the ejaculate, i.e., at a time about 2, 3, 4, 5, 6, 7, 8, 9, 10, 11, 12, 13, 14, 15, 16, 17, 18, 19, 20, 21, 22, 23, or 24 hours after collection of the ejaculate. Preferably the fractionation is carried out between about 2 hours and about 12 hours after collection of the ejaculate. More preferably, the fractionation is carried out between about 4 hours and about 8 hours after collection of the ejaculate. Still more preferably, the fractionation is carried out at about 6 hours after collection of the ejaculate.

During these experiments, it also was determined that the temperature at which an ejaculate or population of spermatozoa is stored from the time of collection until the time of separation (up and including the separation process) had an unexpected effect on the ability of an antibody to bind preferentially to Y-chromosome bearing spermatozoa. Based on observations of the conditions at which experiments were conducted, room temperature may be less favored for storage of an ejaculate prior to fractionation. Thus the invention provides methods for fractionating an ejaculate (or separating spermatozoa determinative of male and female offspring) by fractionating the ejaculate after storage of the ejaculate at less than about 20° C. Preferably, the ejaculate is stored at less than about 19° C., 18° C., 17° C., or 16° C., more preferably less than about 15° C., 14° C., 13° C., or 12° C., still more preferably less than about 11° C., 10° C., 9° C., or 8° C., and yet more preferably less than about 7° C., 6° C., 5° C., or 4° C.

Thus, methods employing this information are provided. The methods for fractionating ejaculates utilizing these time and/or temperature considerations can be performed using the magnetic separator device and the various methods described herein. The methods for fractionating ejaculates with time and/or temperature considerations also can be performed using other separation technologies, as these unexpected properties of spermatozoa are not limited to the magnetic separation technology described herein. This aspect of the invention also provides populations of spermatozoa separated with an understanding of the unexpected properties of time and temperature, methods for artificial insemination using such populations of spermatozoa, and other methods and products that are described more fully herein.

### EXAMPLES

### Example 1

### Separation of Spermatozoa Using the Magnetic Separator

Magnetic beads made by a co-precipitation process and coated with protein A were used. To prepare “bridge-bound beads,” beads were bound to an excess of rabbit anti-mouse IgM bridge antibody for 2 hours, washed magnetically 5× and re-suspended into phosphate buffered saline (PBS). Magnetic washing was performed by placing the suspension of magnetic beads into a dipole magnetic separator for 5 minutes to pull the beads to the walls of the tube, aspirating the clear supernatant from the tube, and resuspending the magnetic beads in 10 ml of PBS.

An aliquot (790 μl) of a freshly collected ejaculate was diluted to 8.0 ml with PBS and then washed 2 times by pelleting by centrifugation at 900×g for ten minutes and resuspension in PBS.

Washed cells were re-suspended in 6.0 ml of PBS and 360 μg of a primary sexing antibody added (Koo et al., Hum. Genet. 58(1):18-20, 1981; U.S. Pat. No. 4,680,258 to Hammerling et al.). The primary sexing antibody recognizes a surface marker found predominately on male (Y chromosome bearing) spermatozoa. The sample was allowed to bind for thirty minutes at room temperature with gentle mixing. Bound cells were washed 1× by pelleting by centrifugation at 900×g for ten minutes.

Washed cells were re-suspended in 6.0 ml of PBS and 1.2 ml of bridge bound magnetic beads were added to bring the solution to 0.2 mg beads/ml solution. Sample was allowed to bind for thirty minutes at room temperature with gentle mixing.

The sample then was placed in the magnetic separator of the invention and the beads were pulled for ten minutes.

Female cells (X chromosome bearing spermatozoa) were retrieved from the bottom of the device by opening the stopcock and draining in a controlled flow.

Cells were then characterized by measuring cell count and motility of the cells cluted from the magentic separator device. A summary of the cell numbers and motility before and after processing with the magnetic separator device is provided in Table 1 below.

TABLE 1

| Property | Value |
|---|---|
| Volume of semen sample | 0.79 ml |
| Cell concentration in ejaculate | 1253 × 10⁶ |
| Total cells into separation | 990 × 10⁶ |
| Motility of cells immediately post | 75% |
| ejaculation | |
| Cell concentration into separator device | 116 × 10⁶ |
| Cell concentration of the eluate from | 77 × 10⁶ |
| separator device | |
| % of input cells recovered from device | 66.4% |
| Motility of the recovered spermatozoa | 75% |

Collected cells were then extended using an egg yolk extender. Commercially available extenders that can be used include Biladyl®, Triladyl® and Biociphos Plus™. The cells then were transferred to straws and frozen using standard freezing techniques.

Frozen straws were thawed approximately 2 months post-freezing and the semen used to produce 109 embryos via in vitro fertilization.

The sex of each individual embryo was determined by PCR amplification of the ZFX and ZFY regions of the X and Y chromosomes respectively. The embryo PCR protocol was used as described in Kirkpatrick and Monson, J. Reprod. Fertil. 98:335-340 (1993), with a minor modification.

All embryos (from 8-cell to hatched blastocyst stage) were produced by in vitro fertilization (IVF). A few embryonic cells were extracted from these embryos and put into a 250 μl PCR tube containing 5 μl lysis buffer (2% 2-mercaptoethanol, 0.01% SDS, 10 mM EDTA, 10 mM Tris pH 8.3, proteinase K, 222 μg/ml). All embryonic cells were lysed at 55° C. for 2 hr, and proteinase K was inactivated at 98° C. for 10 min. Then, the sample was ready for PCR sexing.

The first round PCR was done using primers complementary to both ZFX and ZFY genes (forward primer: ATAATCACATGGAGAGCCACAAGCT (SEQ ID NO:1)); reverse primer: GCACTTCTTTGGTATCTGAGAAAGT (SEQ ID NO:2)). Nested PCR was used to specifically amplify ZFX or ZFY gene using the allele-specific primers (for ZFX, forward primer: GACAGCTGAACAAGTGTTACTG (SEQ ID NO:3)), reverse primer: AATGTCACACTTGAATCGCATC (SEQ ID NO:4)); for ZFY, forward primer: GAAGGCCTTCGAATGTGATAAC (SEQ ID NO:5)), reverse primer: CTGACAAAAGGTGGCGATTTCA (SEQ ID NO:6)). The primers for nested PCR were used to amplify non-overlapping regions of either ZFX or ZFY gene, and to generate 247 bp (ZFX) and 167 bp (ZFY) products. The PCR reactions consisted of 1×GeneAmp® PCR Gold buffer (15 mM Tris-HCl, pH 8.0, 50 mM KCl), 2.5 mM MgCl₂, 45 μM dNTP each, 250 nM of each primer and 1 unit of AmpliTaq Gold DNA polymerase (Applied Biosystems, Foster City, Calif.) in a 50 μl reaction volume. The first round PCR was done by hot-start at 94° C. for 10 min and 5 cycles of denaturation at 94° C. for 1 min, annealing at 55° C. for 1 min and extension at 72° C. for 1 min, and followed by 25 cycles of denaturation at 94° C. for 20 seconds, annealing at 55° C. for 20 seconds, extension at 72° C. for 30 seconds, and final extension at 72° C. for 10 min.

A 2 μl aliquot of the first round PCR products was used for the nested PCR. ZFX and ZFY were amplified in separated tubes, and the cycling protocol was performed in two stages with different annealing temperatures. The annealing temperature of the first five PCR cycles was 52° C., and for the remaining 25 PCR cycles was 60° C. The nested PCR was also done by hot-start at 94° C. for 10 min, with a total of 30 triphasic cycles of denaturation at 94° C. for 1 min, annealing at the temperature described above for 45 seconds and extension at 72° C. for 1 min and with a final extension for 5 min. Following amplification, a 7 μl aliqout of PCR products was mixed with 2 μl of loading buffer (20% Ficoll 400, 1% SDS and 0.25% Xylene Cyanol in 0.1 M Na₂EDTA, pH 8) and was loaded onto 1.5% (W/V) NuSieve agarose gel containing ethidium bromide (0.5 μg/ml). The PCR products were resolved in Tris-acetate EDTA buffer by electrophoresis for 45 min at 82V, and visualized by an UV transilluminator mounted with camera.

The results of the PCR sex determination of the IVF embryos indicated that there were 85 female embryos and 24 male embryos, for an apparent sex bias of 78% in favor of females.

### Example 2

### Effect of Time and Temperature on Efficiency of Separation

In conducting a number of ejaculate fractionations, a variability in the sex bias of offspring was noticed. For some experiments, sex bias strongly favored females (as in Example 1), while in other experiments, the sex bias only weakly favored females.

To determine what factors favored the stronger sex bias, the various parameters of the entire process of ejaculate fractionation were evaluated, including ejaculate collection, storage, shipping and separation of spermatozoa.

Surprisingly, it was found that the time between ejaculate collection and fractionation resulted in a substantial difference in the efficiency of fractionation and the resulting female sex bias in offspring. In accordance with standard practice in the art, ejaculates were used and/or processed as soon as possible after collection. During various fractionation experiments, the time between collection and processing varied. It was determined that the shortest time lag resulted in the least fractionation, i.e., the least amount of separation of X chromosome bearing spermatozoa from the total ejaculate. In addition, it was observed that after 24 hours spermatozoa did not fractionate well because the antibody bound to almost all cells regardless of chromosomal content.

The fractionation results were analyzed by single-cell PCR and by ultrasound of pregnancies from in vitro fertilizations using fractionated spermatozoa. Ultrasound data for a fractionation performed less than about 1 hour post collection showed no sex bias. The ultrasound data suggested a sex bias of 60 to 65% females in fractionations performed at 2.5 hours post-collection. Data from PCR and IVF showed a 71% female bias using spermatozoa from fractionations performed at about 6 hours post-collection.

Therefore, a distinct and surprising increase in the fractionation of X chromosome and Y chromosome bearing spermatozoa was observed in a window of time following collection of the ejaculates. Without wishing to be held to any specific theory, it is believed that the difference in fractionation upon storage represents an increase in the ability of the antibody used to recognize an antigen, possibly by increased access of the antibody to the antigen on the cell surface. Thus, it is believed that the window of preferential separation may represent a changing access of the antibody to the antigen on the spermatozoa cell surface. According to this theory, the access of the antibody to the antigen is greater for Y-bearing spermatozoa within the window than it is for X-bearing spermatozoa. Other antigens are believed to have the same time dependence. The “window” of fractionation appears to open at about 2 hours and to close by less than about 24 hours. It should be noted that this notion of a window of preferential separation of spermatozoa is also applicable to non-magnetic methods of cell separation.

In addition, the temperature at which ejaculates were stored also was an unexpected factor in the efficiency of fractionation and sex bias. During trials of the separation methods, semen samples initially were shipped cold. Later during the testing of the separation methods, semen samples started being shipped at room temperature; at this point the separations became much worse, with little or no sex bias observed.

In general, based on the time and temperature effects on semen fractionation, it appears that time, temperature and/or other parameters causes an increase in the access and/or recognition of the surface antigens needed for efficient separation.

Modifications and improvements within the scope of this invention will occur to those, skilled in the art. The above description is intended to be exemplary only. The scope of this invention is defined only by the following claims and their equivalents.

All patent and literature references disclosed herein are incorporated by reference in their entirety.

What is claimed is:

1. A magnetic separator for separating magnetic components from a test sample that includes the magnetic components and non-magnetic components, the magnetic separator comprising:
a container constructed and arranged to receive the test sample, the container including an inlet and an outlet, the test sample to be received through the inlet;
at least one magnet adapted to generate a magnetic field within the container, the magnetic field to be operative upon the magnetic components within the test sample to substantially separate the magnetic and non-magnetic components from one another; and
a regulator coupled to the outlet of the container to regulate flow of the non-magnetic components from the outlet of the container.

2. The magnetic separator of claim 1, wherein the regulator is actuatable between a closed position and an open position to control the flow of the non-magnetic components from the outlet.

3. The magnetic separator of claim 2, wherein the regulator is actuatable to vary the rate of flow of the non-magnetic components from the outlet.

4. The magnetic separator of claim 1, wherein the regulator includes a valve.

5. The magnetic separator of claim 4, wherein the valve includes a stopcock.

6. The magnetic separator of claim 1, wherein the outlet is located below the inlet.

7. The magnetic separator of claim 6, wherein the outlet of the container is provided at a bottom of the container.

8. The magnetic separator of claim 7, wherein the bottom of the container has a substantially conical shape.

9. The magnetic separator of claim 1, wherein at least a portion of the container is substantially transparent such that the test sample within the container is visible from outside the container.

10. The magnetic separator of claim 1, wherein the at least one magnet includes a bar magnet.

11. The magnetic separator of claim 1, wherein the at least one magnet includes a pair of magnets that are spaced apart about the container.

12. The magnetic separator of claim 11, wherein the magnets are substantially equally spaced about the container.

13. The magnetic separator of claim 12, wherein the magnets are spaced approximately 180° apart about the container.

14. The magnetic separator of claim 1, wherein the magnet is formed of a material selected from the group consisting of neodymium iron boron, samarium cobalt, alnico and ferrite.

15. The magnetic separator of claim 1, wherein the magnet includes at least one electromagnet.

16. The magnetic separator of claim 1, further comprising at least one retainer constructed and arranged to hold the container adjacent the magnet.

17. The magnetic separator of claim 16, wherein the at least one retainer slidably receives the container.

18. The magnetic separator of claim 16, wherein the at least one retainer includes a channel constructed and arranged to receive a portion of an outer surface of the container.

19. A magnetic separator for separating magnetic components from a test sample that includes the magnetic components and non-magnetic components, the magnetic separator comprising:
a container-receiving region that is constructed and arranged to receive a container that is adapted to receive the test sample;
at least two magnets spaced about the container-receiving region, the magnets adapted to generate a magnetic field within the container-receiving region; and
a guide constructed and arranged to position the container within the container-receiving region at a substantially equal distance from each magnet.

20. The magnetic separator of claim 19, wherein the guide includes at least one retainer constructed and arranged to hold the container.

21. The magnetic separator of claim 19, wherein the guide includes at least one channel constructed and arranged to receive at least a portion of an outer surface of the container.

22. The magnetic separator of claim 19, wherein the magnets are substantially equally spaced about the container-receiving region.

23. The magnetic separator of claim 19, wherein the guide is constructed and arranged to slidably receive the container.

24. The magnetic separator of claim 19, wherein the guide is constructed and arranged to receive the container by a snap-fit configuration.

25. The magnetic separator of claim 19, wherein the magnets are formed of a material selected from the group consisting of neodymium iron boron, samarium cobalt, alnico and ferrite.

26. The magnetic separator of claim 19, in combination with the container, the container being positioned in the container-receiving region by the guide at a substantially equal distance from each magnet.

27. A magnetic separator for separating magnetic components from a test sample that includes the magnetic components and non-magnetic components, the separator comprising:
a container-receiving region that is constructed and arranged to receive a container that is adapted to receive the test sample;
at least one magnet disposed adjacent the container-receiving region, the magnet adapted to generate a magnetic field within the container-receiving region, the magnetic field to be operative upon the magnetic components in the test sample; and
a base supporting the container-receiving region above a vessel-receiving region that is constructed and arranged to receive a vessel below the container-receiving region, the vessel adapted to capture the non-magnetic components of the test sample from the container.

28. The magnetic separator of claim 27, wherein the base includes a plurality of legs adapted to elevate the container-receiving region.

29. The magnetic separator of claim 27, wherein the base is securable to a surface.

30. The magnetic separator of claim 27, further comprising at least one retainer constructed and arranged to hold the container in the container-receiving region.

31. The magnetic separator of claim 30, wherein the retainer maintains the magnet spaced a distance from the container-receiving region.

32. The magnetic separator of claim 27, wherein the at least one magnet includes a pair of magnets that are substantially equally spaced about the container-receiving region.

33. The magnetic separator of claim 27, wherein the at least one magnet includes a bar magnet.

34. The magnetic separator of claim 27, wherein the magnet is formed of a material selected from the group consisting of neodymium iron boron, samarium cobalt, alnico and ferrite.

35. The magnetic separator of claim 27, wherein the magnet includes at least one electromagnet.

36. The magnetic separator of claim 27, in combination with the container, the container being positioned in the container-receiving region and being adapted to receive the test sample.

37. The magnetic separator of claim 36, in combination with the vessel, the vessel being positioned in the vessel-receiving region and adapted to capture the non-magnetic components from the container.

38. The magnetic separator of claim 37, wherein the container includes an outlet constructed and arranged for the non-magnetic components to flow out of the container from the outlet.

39. The magnetic separator of claim 38, wherein the vessel is provided to receive the flow of the non-magnetic components from the outlet of the container.

40. The magnetic separator of claim 38, wherein the outlet includes a regulator to regulate flow of the non-magnetic components from the outlet of the container.

41. A method for magnetically separating a selected population of cells from a biological sample, comprising contacting the biological sample in a container with a plurality of binding agent molecules that selectively bind the selected population of cells, for a time sufficient for the binding agent molecules to bind the cells, wherein the binding agent molecules are attached to magnetic particles, to form a magnetic component of the biological sample;
applying an external magnetic field to the container to separate the magnetic component from the non-magnetic components of the biological sample; and
draining the non-magnetic components of the biological sample from the container to separate the selected population of cells from the non-magnetic components of the biological fluid sample.

42. The method of claim 41, wherein the biological sample comprises a second population of cells and wherein the non-magnetic components of the biological fluid sample comprise the second population of cells.

43. The method of claim 41, wherein the binding agent molecule is an antibody or antigen-binding fragment thereof.

44. The method of claim 43, wherein the antibody is specific for Y-bearing sperm.

45. The method of claim 43, wherein the antibody is specific for X-bearing sperm.

46. The method of claim 43, wherein the antibody is attached to the magnetic particles through an intermediate linking compound.

47. The method of claim 46, wherein the intermediate linking compound is Protein A.

48. The method of claim 41, wherein the binding agent molecule is a phage display binding molecule.

49. The method of claim 41, wherein the binding agent molecule is a lectin.

50. The method of claim 41, wherein the binding agent molecule is a binding partner of a molecule on the cell.

51. The method of claim 41, wherein the magnetic particle is a non-porous magnetic bead support having a diameter of 0.1 to 2 microns.

52. The method of claim 41 or claim 51, wherein the magnetic particle is covalently attached to the binding agent molecule.

53. The method of claim 41, wherein the selected population of cells is spermatozoa determinative of one sex.

54. The method of claim 41, wherein the magnetic field is insufficient to hold the magnetic particles to the surface of the container.

55. The method of claim 54, wherein the selected population of cells bound to the magnetic particles form a phase separate from the remainder of the biological fluid sample.

56. The method of claim 54, wherein the selected population of cells bound to the magnetic particles form a bolus upon draining that protrudes from the interior surface of the container.

57. The method of claim 41, wherein the magnetic particles are too numerous to form a monolayer of particles on the walls of the container under the influence of the magnetic field.

58. The method of claim 41, wherein the number of cells in the selected population of cells is greater than about 1×10⁵ cells/ml.

59. The method of claim 41, further comprising removing the selected population of cells from the container.

60. The method of claim 59, wherein the step of draining the selected population of cells from the container comprises draining the container by gravity.

61. The method of claim 60, wherein the step of draining is regulated by opening and optionally closing a valve or stopcock, or regulating the operation of a pump attached to a drain.

62. The method of claim 59, wherein the step of draining the selected population of cells from the container comprises pumping a dense fluid into the container to displace the non-magnetic components of the biological sample from the container.

63. A method of insemination comprising
obtaining a population of spermatozoa according to the method of claim 53, and
inseminating a mammal with the population of spermatozoa.

64. A method for magnetically separating a selected population of cells from a biological sample, comprising
contacting the biological fluid sample with a binding agent that selectively binds the selected population of cells for a time sufficient for the binding agent to bind the selected population of cells to form a reaction mixture, wherein the binding agent is attached to a magnetic particle;
transferring the reaction mixture to a separation container;
applying an external magnetic field to the separation container to separate the magnetic particles from the biological fluid sample; and
draining the non-magnetic components of the biological sample from the container to separate the selected population of cells from the non-magnetic components of the biological fluid sample.

65. The method of claim 64, wherein the biological sample comprises a second population of cells and wherein the non-magnetic components of the biological fluid sample comprise the second population of cells.

66. The method of claim 64, wherein the binding agent molecule is an antibody or antigen-binding fragment thereof.

67. The method of claim 66, wherein the antibody is specific for Y-bearing sperm.

68. The method of claim 66, wherein the antibody is specific for X-bearing sperm.

69. The method of claim 66, wherein the antibody is attached to the magnetic particles through an intermediate linking compound.

70. The method of claim 69, wherein the intermediate linking compound is Protein A.

71. The method of claim 64, wherein the binding agent molecule is a phage display binding molecule.

72. The method of claim 64, wherein the binding agent molecule is a lectin.

73. The method of claim 64, wherein the binding agent molecule is a binding partner of a molecule on the cell.

74. The method of claim 64, wherein the magnetic particle is a non-porous magnetic bead support having a diameter of 0.1 to 2 microns.

75. The method of claim 64 or claim 74, wherein the magnetic particle is covalently attached to the binding agent molecule.

76. The method of claim 64, wherein the selected population of cells is spermatozoa determinative of one sex.

77. The method of claim 64, wherein the magnetic field is insufficient to hold the magnetic particles to the surface of the container.

78. The method of claim 77, wherein the selected population of cells bound to the magnetic particles form a phase separate from the remainder of the biological fluid sample.

79. The method of claim 77, wherein the selected population of cells bound to the magnetic particles form a bolus upon draining that protrudes from the interior surface of the container.

80. The method of claim 64, wherein the magnetic particles are too numerous to form a monolayer of particles on the walls of the container under the influence of the magnetic field.

81. The method of claim 64, wherein the number of cells in the selected population of cells is greater than about 1×10⁵ cells/ml.

82. The method of claim 64, further comprising removing the selected population of cells from the container.

83. The method of claim 82, wherein the step of draining the selected population of cells from the container comprises draining the container by gravity.

84. The method of claim 83, wherein the step of draining is regulated by opening and optionally closing a valve or stopcock, or regulating the operation of a pump attached to a drain.

85. The method of claim 82, wherein the step of draining the selected population of cells from the container comprises pumping a dense fluid into the container to displace the non-magnetic components of the biological sample from the container.

86. A method of insemination comprising
obtaining a population of spermatozoa according to the method of claim 76, and
inseminating a mammal with the population of spermatozoa.

87. A method of increasing the percentage of mammalian offspring of either sex, comprising
magnetically separating spermatozoa determinative of one sex from a biological sample containing spermatozoa of determinative of both sexes by:
(a) contacting the biological fluid sample in a container with a plurality of binding agent molecules that selectively bind the spermatozoa determinative of one sex, for a time sufficient for the binding agent molecules to bind the spermatozoa determinative of one sex, wherein the binding agent molecules are attached to magnetic particles;
(b) applying an external magnetic field to the container to separate the magnetic particles from the remainder of the biological fluid sample containing spermatozoa determinative of the other sex; and
(c) draining by gravity the remainder of the biological fluid sample from the container to separate the spermatozoa determinative of one sex from the remainder of the biological fluid sample containing the spermatozoa determinative of the other sex, then administering spermatozoa determinative of the other sex to the reproductive tract of a female mammal.

88. The method of claim 87, further comprising washing the spermatozoa determinative of the other sex prior to administering the spermatozoa to the reproductive tract of a female mammal.

89. The method of any of claims 87-88, wherein the step of administering is artificial insemination.

90. The method of any of claims 87-88, wherein the mammal is selected from the group consisting of cattle, sheep, pigs, goats, horses, dogs and cats.

91. The method of any of claims 87-88, wherein the number of spermatozoa administered is at least about 10 million.

92. The method of claim 91, wherein the number of spermatozoa administered is at least about 20 million.

93. The method of claim 92, wherein the number of spermatozoa administered is at least about 30 million.

94. The method of claim 93, wherein the number of spermatozoa administered is at least about 40 million.

95. The method of claim 94, wherein the number of spermatozoa administered is at least about 50 million.

96. The method of any of claims 87-88, wherein the wherein the number of spermatozoa administered is less than about 10 million.

97. The method of claim 96, wherein the number of spermatozoa administered is less than about 1 million.

98. The method of claim 97, wherein the number of spermatozoa administered is less than about 0.5 million.

99. The method of claim 87, wherein the biological sample contains greater than about 1×10⁵ cells/ml.

100. The method of claim 87, wherein the binding agent molecules that selectively bind the spermatozoa determinative of one sex are antibodies.

101. The method of claim 87, wherein the antibodies are specific for Y-bearing sperm.

102. The method of claim 101, wherein the antibodies are specific for an H—Y antigen.

103. The method of claim 87, wherein the antibodies are specific for X-bearing sperm.

104. The method of claim 101 or 103, wherein the antibodies are monoclonal antibodies.

105. The method of claim 87, wherein the magnetic particles have a diameter of 0.1 to 0.5 microns.

106. A method for fractionating an entire ejaculate of a mammal in a single process, comprising
obtaining an ejaculate, and
subjecting the ejaculate to the method of claim 53 or 76.

107. The method of claim 106, wherein the ejaculate is fractionated with an efficiency of at least about 55%.

108. The method of claim 107, wherein the ejaculate is fractionated with an efficiency of at least about 56%.

109. The method of claim 108, wherein the ejaculate is fractionated with an efficiency of at least about 57%.

110. The method of claim 109, wherein the ejaculate is fractionated with an efficiency of at least about 58%.

111. The method of claim 110, wherein the ejaculate is fractionated with an efficiency of at least about 60%.

112. The method of claim 111, wherein the ejaculate is fractionated with an efficiency of at least about 65%.

113. The method of claim 112, wherein the ejaculate is fractionated with an efficiency of at least about 70%.

114. The method of claim 113, wherein the ejaculate is fractionated with an efficiency of at least about 75%.

115. The method of claim 114, wherein the ejaculate is fractionated with an efficiency of at least about 80%.

116. The method of claim 115, wherein the ejaculate is fractionated with an efficiency of at least about 85%.

117. The method of claim 116, wherein the ejaculate is fractionated with an efficiency of at least about 90%.

118. The method of claim 117, wherein the ejaculate is fractionated with an efficiency of at least about 95%.

119. The method of claim 118, wherein the ejaculate is fractionated with an efficiency of at least about 99%.

120. A method of insemination comprising
obtaining a mammalian ejaculate,
fractionating the ejaculate according to the method of claim 53 or 76 to obtain a population of spermatozoa, and
inseminating a mammal with the population of spermatozoa.

121. The method of claim 120, wherein the conception rate of offspring resulting from the insemination is at least about 50% of the conception rate obtained using unfractionated spermatozoa.

122. The method of claim 120, wherein the conception rate of offspring resulting from the insemination is at least about 70% of the conception rate obtained using unfractionated spermatozoa.

123. The method of claim 120, wherein the conception rate of offspring resulting from the insemination is at least about 80% of the conception rate obtained using unfractionated spermatozoa.

124. The method of claim 120, wherein the conception rate of offspring resulting from the insemination is at least about 90% of the conception rate obtained using unfractionated spermatozoa.

125. The method of claim 120, wherein the conception rate of offspring resulting from the insemination is at least about 95% of the conception rate obtained using unfractionated spermatozoa.

126. A method for creating a sex bias in mammalian offspring, comprising
obtaining a population of spermatozoa from an ejaculate fractionated according to the method of claim 106, and
inseminating a mammal with the population of spermatozoa.

127. The method of claim 106, wherein the ejaculate is fractionated in less than about 2 hours.

128. The method of claim 127, wherein the ejaculate is fractionated in less than about 1 hour.

129. A method for fractionating spermatozoa of a mammal without a substantial loss of motility, comprising
obtaining an ejaculate containing spermatozoa, and
subjecting the ejaculate to the method of claim 53 or 76.

130. The method of claim 129, wherein motility of the fractionated spermatozoa is at least about 50% of the unprocessed spermatozoa.

131. The method of claim 130, wherein motility of the fractionated spermatozoa is at least about 60% of the unprocessed spermatozoa.

132. The method of claim 131, wherein motility of the fractionated spermatozoa is at least about 70% of the unprocessed spermatozoa.

133. The method of claim 132, wherein motility of the fractionated spermatozoa is at least about 80% of the unprocessed spermatozoa.

134. The method of claim 133, wherein motility of the fractionated spermatozoa is at least about 90% of the unprocessed spermatozoa.

135. The method of claim 134, wherein motility of the fractionated spermatozoa is at least about 95% of the unprocessed spermatozoa.

136. The method of claim 135, wherein motility of the fractionated spermatozoa is at least about 97% of the unprocessed spermatozoa.

137. The method of claim 136, wherein motility of the fractionated spermatozoa is at least about 98% of the unprocessed spermatozoa.

138. The method of claim 137, wherein motility of the fractionated spermatozoa is at least about 99% of the unprocessed spermatozoa.

139. A population of fractionated spermatozoa determinative of one sex wherein at least about 50% of the spermatozoa are motile.

140. The population of fractionated spermatozoa of claim 139, wherein at least about 60% of the spermatozoa are motile.

141. The population of fractionated spermatozoa of claim 140, wherein at least about 70% of the spermatozoa are motile.

142. The population of fractionated spermatozoa of claim 141, wherein at least about 80% of the spermatozoa are motile.

143. The population of fractionated spermatozoa of claim 142, wherein at least about 85% of the spermatozoa are motile.

144. The population of fractionated spermatozoa of claim 143, wherein at least about 90% of the spermatozoa are motile.

145. The population of fractionated spermatozoa of claim 144, wherein at least about 95% of the spermatozoa are motile.

146. The population of fractionated spermatozoa of claim 145, wherein at least about 97% of the spermatozoa are motile.

147. The population of fractionated spermatozoa of claim 146, wherein at least about 98% of the spermatozoa are motile.

148. The population of fractionated spermatozoa of claim 147, wherein at least about 99% of the spermatozoa are motile.

149. A method for fractionating an ejaculate of a mammal, comprising
obtaining an ejaculate, and
fractionating the ejaculate between about 2 hours and about 24 hours after collection of the ejaculate.

150. The method of claim 149, wherein the fractionation is carried out between about 2 hours and about 12 hours after collection of the ejaculate.

151. The method of claim 150, wherein the fractionation is carried out between about 4 hours and about 8 hours after collection of the ejaculate.

152. The method of claim 151, wherein the fractionation is carried out at about 6 hours after collection of the ejaculate.

153. A method for fractionating an ejaculate of a mammal, comprising
obtaining an ejaculate, and
fractionating the ejaculate after storage of the ejaculate at less than about 20° C.

154. The method of claim 153, wherein the fractionation is carried out after the ejaculate is stored at less than about 16° C.

155. The method of claim 154, wherein the fractionation is carried out after the ejaculate is stored at less than about 12° C.

156. The method of claim 155, wherein the fractionation is carried out after the ejaculate is stored at less than about 8° C.

157. The method of claim 156, wherein the fractionation is carried out after the ejaculate is stored at less than about 4° C.
